(19) United States
(12) Patent Application Publication
(10) Pub. No.: US 2003/0165870 A1
(43) Pub. Date: Sep. 4, 2003

(54) Novel sequences of E. coli CFT073

(76) Inventors: Frederick R. Blattner, Madison, WI (US); Rodney A. Welch, Madison, WI (US); Valerie D. Burland, Cross Plains, WI (US)

Correspondence Address: Nicholas J. Seay, One South Pinckney Street, Stuite 600, P.O. Box 2113, Madison, WI, 53701-2113, US

(21) Appl. No.: 10/085,959

(22) Filed: Mar. 1, 2002

(51) Int. Cl. C12Q 1/68; C07H 21/04; C12N 1/21

(52) U.S. Cl. 435/6; 435/252.33; 536/23.7

(57) ABSTRACT

The entire genome of pathogenic E. coli strain CFT073 has been sequenced. Nearly all of the genomic DNA sequences present in CFT073 and absent in the previously sequenced laboratory strain K-12 are presented here.

### STATEMENT REGARDING FEDERALLY SPONSORED RESEARCH OR DEVELOPMENT

This invention was made with United States government support awarded by the following agency: NIH A144387. The United States has certain rights in this invention.

### CROSS-REFERENCE TO RELATED APPLICATION

Not applicable.

### BACKGROUND OF THE INVENTION

Escherichia coil is a common enteric bacterial strain that has both laboratory and human health importance. One particular strain of E. coli, designated CFT073 is a human pathogen that causes urinary tract infections. Urinary tract infections are common in various populations throughout the human life span. Infant boys, women of childbearing years, and aged people of both sexes have relatively high incidences of this infection. Acute pyelonephritis, a bacterial infection of the kidneys, is a common complication of such infections. Acute pyelonephritis often requires hospitalization for treatment, and the disease can be severe including complications or life threatening conditions.

Various strains of E. coil are associated with urinary tract infections and are commonly found in the urine of patients with pyelonephritis. Certain phenotypes of E. coil are found more often in such association. The strains associated with acute pyelonephritis often include a set of gene functions which, as a unit, have been thought to form a set of virulence factors that allow specific clones of E. coli to cause pyelonephritis.

Accordingly to investigate diagnosis or treatment of this disease, it is appropriate to focus the inquiry on the presumptive virulence factors. Most, if not all, of those virulence factors are present in the strain CFT073, which is known to be the among the most virulent of all of the E. coil strains associated with these diseases. The strain CFT073 has been previously shown to contain a pathogenicity island associated with uropathogenicity. Kao, JS et al., Infect Immun. 65:7, pp.2812-2920 (1997).

Modem geneticists have been working to resolve the genetic code of many organisms. Great efforts have been made to sequence the human genome. The effort to sequence the genomes of whole organisms began with an effort to sequence the genome of E. coli. For the original effort to sequence the E. coli genome, a useful and common laboratory strain, designated K-12, was chosen. The entire genome of that strain was sequenced and published. Science, 277:1453-1462 (1997). Since the genes which are responsible for the pathogenicity of E. coli CFT073 are missing from strain K-12, the sequence of the K-12 genome is of limited help in developing tools to detect, hinder or destroy E. coli CFT073.

### BRIEF SUMMARY OF THE INVENTION

It is an object of the present invention to provide the DNA sequence present in E. coli CFT073 which is not present in non-pathogenic E. coli to enable detection, diagnosis, prophylaxis and therapeutic tools to combat bacterial infections.

It is another object of the present invention to provide a means to detect E. coli CFT073 in an infection of an environmental sample.

It is yet another object of this invention to provide a means for the early diagnosis of humans and livestock infected with CFT073.

Another object of the present invention is to provide a means of treating humans and livestock infected with CFT073.

It is a further object of the present invention to provide a means for the prevention of infection by CFT073.

The present invention includes many DNA sequences that are unique to E. coli CFT073.

One aspect of the present invention is two CFT073 DNA sequences that encode hemagglutinin-like proteins that are important for host cell adhesion.

Another aspect of the present invention is two CFT073 DNA sequences that encode for autotransporters.

Another aspect of the present invention is a CFT073 DNA sequence that encodes for a RTX-like protein.

Still another aspect of the present invention is a method for detecting E. coli CFT073 and distinguishing the strain from other strains of E. coli by genetic analysis and testing.

It is a feature of the invention disclosed here that virtually the entire genome of E. coli CFT073 is set forth in the data contained here, combined with the information already published in the field.

### BRIEF DESCRIPTION OF THE SEVERAL VIEWS OF THE DRAWINGS

Not applicable.

### DETAILED DESCRIPTION OF THE INVENTION

The investigators here have sequenced virtually the entire genome of E. coli CFT073. Presented in this specification is essentially all the DNA sequence which is contained in the genome of E. coli strain CFT073 and not found in the previously sequenced non-pathogenic laboratory E. coli strain K-12. The genome sequence is essentially complete, lacking only an occasional presumably small sequence linkage between established long sequences known. The availability of the sequence data presented here will enable intelligent design of diagnostic detection, prophylaxis and therapeutic tools for disease and infections caused by this organism.

The sequence of E. coli CFT073 was, in brief, performed by shotgun cloning and duplicative random sequence analysis followed by computer assembly into contigs. The contigs determined by shot gun clone sequencing were assembled using computer software designed for that purpose.

An important analysis which has begun on this sequence data is the identification of genetic sequences associated with the pathogenesis of infection, which sequences provide information essential to the diagnosis, treatment, and prevention of infection by uropathogenic E. coli strains. In order to facilitate the identification of genes involved in the pathogenesis of infection by uropathogenic E. coli strains for use in detection of the pathogen, and in the diagnosis, treatment, and prevention of uropathogenic infection, the entire genomic DNA sequence of E. coli CFT073 was compared with that of E. coli K-12, a nonpathogenic laboratory strain as published in Science, 277:1453-62 (1997).

Attached to this patent application is a sequence listing containing essentially all of the DNA sequence in the CFT073 genome, represented by the contigs mentioned above, that are not present in the K-12 genome. This sequence is present in the sequence listing as SEQ ID NO:1 through SEQ ID NO:251 and SEQ ID NO:254.

By definition, the genetic material in the sequences disclosed in this invention is sufficient for pathogenicity in humans since strain CFT073 is highly pathogenic while K-12 is not. In addition, analysis of the open reading frames (ORFs) and computer comparisons to sequences from other pathogens have allowed identification of several of the ORFs which code for proteins specifically associated with pathogenicity. We provide five examples of such ORFs. The first one (ORF1) is between nucleotide 12003 and nucleotide 20509 of SEQ ID NO:25 1. ORF1 is a putative member of the ShlA/HecA/Fha exoprotein family that shows a 25% identity over 2,311 residues to a probable hemagglutinin (the nucleotide sequence GenBank accession number for the probable hemagglutinin is AE004443). The second one (ORF2) is between nucleotide 31940 and nucleotide 34668 of SEQ ID NO:254. ORF2 is a member of the Shl/Fha/Hpm family and amino acids 33-907 of ORF2 shows a 26% identity to amino acids 1,912-2,818 of hemagglutinin/hemolysin-related protein (the amino acid sequence and the nucleotide sequence GenBank accession numbers are AAG03431 and AE002405, respectively). Both ORF1 and ORF2 are believed to be important for host cell adhesion and thus infection.

The third one (ORF3) is the complementary sequence between nucleotide 1008 and nucleotide 1885 of SEQ ID NO:85. ORF3 is a member of the autotransporter family and is 57% identical over 292 residues to a putative beta-barrel outer membrane protein (the nucleotide sequence GenBank accession number is AE005210). The fourth one (ORF4) is the complementary sequence between nucleotide 1996 and nucleotide 5607 of SEQ ID NO:85. ORF4 is also a member of the autotransporter family. ORF4 has a 31% identity over 1103 residues to YapD protein (the nucleotide sequence GenBank accession number is AJ277627) and a 27% identity over 2952 residues to YapH protein (the nucleotide sequence GenBank accession number is AJ277631). Both ORF3 and ORF4 as autotransporters are believed to be important virulence factors of CFT073.

The fifth one (ORF5) is between nucleotide 40329 and nucleotide 44950 of SEQ ID NO:251. ORF5 is similar to RTX family members and is 23% identical over 1,461 residues to a putative RTX family exoprotein (the nucleotide sequence GenBank accession number is AE005229). ORF5 is believed to be an exotoxin like RTX.

In addition to the diagnostic value, the DNA sequences of ORF1-5 are also useful for treatment and prevention purposes. For example, antisense oligonucleotides can be designed given the knowledge of these sequences to block the expression of the corresponding proteins. One of ordinary skill knows how to design and use antisense oligonucleotides. Along the same line, the corresponding amino acid sequences of ORF1-5 or an immunogenic fragment thereof are also valuable for diagnosis, treatment and prevention of uropathogenic E. coli strains. For example, the corresponding amino acid sequences or an immunogenic fragment thereof can be used to generate antibodies that can be used for diagnosis, treatment and prevention purposes. One of ordinary skill in the art knows how to produce antibodies to the proteins encoded by ORF1-5. Vaccines may also be produced using the amino acid sequence information. It is well within the knowledge of a skilled artisan to generate vaccines.

The specific CFT073 strain from which the sequence data is derived is available from ATCC as ATCC 700928. One wishing to practice the present invention using one of the disclosed DNA sequences can do so by isolating the sequence from ATCC 700928 using knowledge of the nucleotide sequence and standard methods known to one of ordinary skill in the art.

It is expected that minor sequence variations in E. coli CFT073-specific nucleotide sequences associated with nucleotide additions, deletions, and mutations, whether naturally occurring or introduced in vitro, would not interfere with the usefulness of these sequences in the detection of uropathogenic E. coli, in methods for preventing urinary tract infection, and in methods for treating pyelonephritis. Therefore, the scope of the present invention is intended to encompass minor variations in the claimed sequences, which include both DNA and RNA and can also contain non-standard bases such as inosine.

Another utility enabled by the disclosure here is the detection of pathogenic E. coli strains by nucleic acid hybridization assays. Such assays, using techniques well known in the art, are made possible by the sequence information contained here, which enables the selection of CFT073-specific probes. By an E. coli CFT073-specific nucleotide probe, it is meant a sequence that is able to hybridize to E. coli CFT073 target DNA present in a sample containing E. coli CFT073 under suitable hybridization conditions and which does not hybridize with DNA from other E. coli strains or from other bacterial species. In particular, a CFT073 specific probe will bind to CFT073 DNA but not to DNA from K-12. This permits the intelligent design of DNA probes for use in hybridization assays for the presence of CFT073 strains. It is well within the ability of one skilled in the art to determine suitable hybridization conditions, based on probe length, G+C content, and the degree of stringency required for a particular application.

The probe may be RNA or DNA. Depending on the detection means employed, the probe may be unlabeled, radiolabeled, or labeled with a dye. The probe may be hybridized with a sample that has been immobilized on a solid support such as nitrocellulose or a nylon membrane, or the probe may be immobilized on a solid support, such as a silicon chip.

The sample to be tested may include blood, urine, feces, or other materials from a human or a livestock animal. Alternatively, the sample may include food intended for human consumption. The sample may be tested directly, or may be treated in some manner prior to testing. For example, the sample may be subjected to PCR amplification using appropriate oligonucleotide primers.

Any means of detecting DNA-RNA or DNA-DNA hybridization known to the art may be used in the present invention.

Again, presented in this specification is a sequence listing constituting essentially all of the DNA sequence in the CFT073 genome that do not appear in strain K-12, which is presented as SEQ ID NO:1 to SEQ ID NO:251 and SEQ ID NO:254. Since all of these sequences are diagnostic of CFT073, as compared to K-12, sequence information from any of these sequences can be used to design diagnostic probes useful to distinguish strain CFT073 from strain K-12 using molecular techniques. To have reasonable assurance of success under conditions of variable stringency, it is preferred that such diagnostic probes use sequences which are at least 25 nucleotides or longer in length. Any 25-mer selected from amongst any of the sequences in any of SEQ ID NO:1 through SEQ ID NO:25 1 and SEQ ID NO:254 may be used for such a probe.

We claim:

1. An isolated nucleic acid molecule comprising a nucleotide sequence or its complement, the nucleotide sequence is identical to at least twenty-five continuous nucleotides contained in DNA sequences selected from the group consisting of SEQ ID NO:1 to SEQ ID NO:251 and SEQ ID NO:254.

2. A recombinant nucleic acid construction comprising the isolated nucleic acid molecule of claim 1.

3. A host cell comprising the recombinant nucleic acid construction of claim 2.

4. A method for distinguishing E. coli bacteria of strain CFT073 from strain K-12 comprising analyzing the genome of the bacteria for the presence of any DNA sequence claimed in claim 1.

5. A method for detecting E. coli CFT073 in a sample comprising the steps of:
a) providing an E. coli CFT073-specific nucleotide probe, the probe comprising a sequence or its complement, the sequence is selected from the group consisting of SEQ ID NO:1 through SEQ ID NO:251 and SEQ ID NO:254;
b) contacting the probe with a sample of test material under suitable hybridization conditions; and
c) detecting hybridization of the probe to a complementary nucleotide sequence in the sample.

6. An isolated nucleic acid molecule comprising a nucleotide sequence or its complement, the nucleotide sequence is selected from the group consisting of the complementary sequence between nucleotide 1008 and nucleotide 1885 of SEQ ID NO:85, the complementary sequence between nucleotide 1996 and nucleotide 5607 of SEQ ID NO:85, the sequence between nucleotide 12003 and nucleotide 20509 of SEQ ID NO:251, the sequence between nucleotide 40329 and nucleotide 44950 of SEQ ID NO:25 1, and the sequence between nucleotide 31940 and nucleotide 34668 of SEQ ID NO:254.

7. A recombinant nucleic acid construction comprising the isolated nucleic acid molecule of claim 6.

8. A host cell comprising the recombinant nucleic acid construction of claim 7.

9. An isolated polypeptide comprising an amino acid sequence encoded by a DNA sequence selected from the group consisting of the complementary sequence between nucleotide 1008 and nucleotide 1885 of SEQ ID NO:85, the complementary sequence between nucleotide 1996 and nucleotide 5607 of SEQ ID NO:85, the sequence between nucleotide 12003 and nucleotide 20509 of SEQ ID NO:251, the sequence between nucleotide 40329 and nucleotide 44950 of SEQ ID NO:25 1, and the sequence between nucleotide 31940 and nucleotide 34668 of SEQ ID NO:254.

10. An isolated polypeptide comprising an immunogenic fragment of an amino acid sequence encoded by a DNA sequence selected from the group consisting of the complementary sequence between nucleotide 1008 and nucleotide 1885 of SEQ ID NO:85, the complementary sequence between nucleotide 1996 and nucleotide 5607 of SEQ ID NO:85, the sequence between nucleotide 12003 and nucleotide 2 0509 of SEQ ID NO:251, the sequence between nucleotide 40329 and nucleotide 44950 of SEQ ID NO:251, and the sequence between nucleotide 31940 and nucleotide 34668 of SEQ ID NO:254.

11. An antibody that specifically binds to the polypeptide of claim 9.
